(19) United States
(12) Patent Application Publication
(10) Pub. No.: US 2008/0025459 A1
(43) Pub. Date: Jan. 31, 2008

(54) X-RAY HYBRID DIAGNOSIS SYSTEM

(76) Inventors: Yilun Shi, Beijing (CN); Jie Jiao, Beijing (CN)

Correspondence Address: Patrick W. Rasche;Armstrong Teasdale LLP, Suite 2600, One Metropolitan Square, St. Louis, MO, 63102, US

(21) Appl. No.: 11/829,572

(22) Filed: Jul. 27, 2007

(30) Foreign Application Priority Data
Jul. 28, 2006 (CN) 200610107654.X

(51) Int. Cl. A61B 6/03

(52) U.S. Cl. 378/10

(57) ABSTRACT

An X-ray radiography unit (103) irradiates a patient with X-rays from a first X-ray tube (127) to obtain an X-ray two-dimensional radiographic image (PI). An X-ray CT unit (101) irradiates the patient with X-rays from a second X-ray tube (125) and acquires projection data, to reconstruct an image using the acquired projection data, thereby obtaining a tomography image (TI). A control unit (50) defines correspondences between position information of the patient located in the X-ray radiography unit and position information of the patient located in the X-ray CT unit. The clearly demonstrated positional correspondence in the X-ray hybrid diagnosis system can facilitate a diagnosis and obviates the need for extra X-raying operations, to thereby ease the strain placed on patients.

### CROSS REFERENCE TO RELATED APPLICATIONS

This application claims the benefit of Chinese Application No. 200610107654.X filed Jul. 28, 2006

### BACKGROUND OF THE INVENTION

This invention relates to an X-ray hybrid diagnosis system which incorporates an X-ray radiography system for obtaining X-ray two-dimensional radiographic (roentgen graphic) images, and a medical X-ray computed tomography (CT) system.

To make a diagnosis upon a patient, depending upon the conditions of the disease or injury of the patient, the X-ray computed radiography (CR) system is used to take X-ray two-dimensional radiographic images, or the X-ray CT system is used to acquire projection data for display of tomography images. Thus, hospitals should normally have the both systems equipped separately, which would disadvantageously involve considerable cost and take up a large footprint.

Moreover, a patient who has been subjected to the X-ray CR system to take X-ray two-dimensional radiographic images may subsequently have to be put through the X-ray CT system to have the tomography images inspected. In such instances, since no positional correspondence is provided between the X-ray two-dimensional radiographic images and the tomography images, it is difficult to identify a part of a subject (patient) in an X-ray two-dimensional radiographic image as the same part of the same subject in a corresponding tomography image, or to identify a part of a subject (patient) in a tomography image as the same part of the same subject in a corresponding X-ray two-dimensional radiographic image. Such a patient may also need to have contrast media administered twice for imaging operations with the X-ray CR system and the X-ray CT system, for which or other reasons, heavy strains would be imposed on the patient. Related techniques hitherto proposed are disclosed for example in JP 8-280666 A.

### SUMMARY OF THE INVENTION

The existing X-ray hybrid diagnosis system having an X-ray radiography system and a medical X-ray computed tomography (CT) system incorporated therein would place an extra burden on an operator such as a radiographer, because no positional correspondence could be provided between the X-ray two-dimensional radiographic images obtained by the X-ray CR system and the tomography images obtained by the X-ray CT system. Therefore, it is an object of the present invention to provide an improved X-ray hybrid diagnosis system in which the positional correspondence between the X-ray two-dimensional radiographic images obtained by the X-ray CR system and the tomography images obtained by the X-ray CT system is provided so that an operator can clearly recognize correspondence in position between/among respective images, for example, with positional correspondence displayed on a monitor or the like, to thereby lessen a burden on the operator in his/her diagnosis task. It is another object of the present invention to provide an X-ray hybrid diagnosis system in which the positional correspondence between X-ray two-dimensional radiographic images obtained by the X-ray CR system and tomography images obtained by the X-ray CT system is clearly demonstrated, thus facilitating a diagnosis and obviating the need for extra X-raying operations, to thereby ease the strain placed on patients.

In a first aspect of the present invention, there is provided an X-ray hybrid diagnosis system which includes an X-ray radiography unit, an X-ray CT unit and a control unit. The X-ray radiography unit irradiates a subject with X-rays from a first X-ray tube to obtain an X-ray two-dimensional radiographic image. The X-ray CT unit irradiates the subject with X-rays from a second X-ray tube and acquiring projection data, to reconstruct an image using the acquired projection data, thereby obtaining a tomography image. The control unit defines correspondences between position information of the subject located in the X-ray radiography unit and position information of the subject located in the X-ray CT unit. The X-ray hybrid diagnosis system consistent with the first aspect of the present invention can establish correspondences between the position information for the X-ray radiography operation and the position information for the X-ray CT scan operation, and thus makes the correspondence in position between the resulting images clearly recognizable, thereby facilitating the diagnosis.

In a second aspect, the X-ray hybrid diagnosis system consistent with the present invention further includes a display unit. The display unit displays the X-ray two-dimensional radiographic image obtained in the X-ray radiography unit. The position information of the subject located in the X-ray CT unit is shown in the displayed X-ray two-dimensional radiographic image. The X-ray hybrid diagnosis system consistent with the second aspect of the present invention is designed to associate the position information for the X-ray CT scan operation with the X-ray two-dimensional radiographic image obtained in the X-ray radiography unit, and thus can utilize the X-ray two-dimensional radiographic image for the X-ray CT scanning or other operation. This can eliminate the necessity to use contrast media for each imaging operation in the X-ray radiography unit and the X-ray CT unit.

In a third aspect, the X-ray radiography unit consistent with the present invention further includes a display unit, as in the second aspect. The display unit displays the tomography image obtained in the X-ray CT unit and the X-ray two-dimensional radiographic image obtained in the X-ray radiography unit concurrently, and the position information of the subject located in the X-ray CT unit is shown in the displayed X-ray two-dimensional radiographic image. In the X-ray hybrid diagnosis system according to the third aspect of the present invention, an operator, for example, can make a diagnosis of an abnormal part of a subject detected in an X-ray two-dimensional radiographic image while observing the same part of the subject located in a tomography image.

In a fourth aspect, the X-ray CT unit consistent with the present invention includes a condition-setting unit in which conditions for obtaining the tomography image are set with the help of the X-ray two-dimensional radiographic image. In order to set the conditions such as a scan range (target part of the subject to be CT-scanned), conventionally, a preliminary X-raying operation is performed in advance to obtain a scout image on which the scan range will be determined. In the X-ray hybrid diagnosis system according to the fourth aspect of the present invention, the position information (usually specified as a position along an axis extending in a predetermined direction) for the X-ray CT scan operation is associated with the X-ray two-dimensional radiographic image obtained in the X-ray radiography unit; therefore, when an X-ray two-dimensional radiographic image is obtained for diagnosis of a specific target part of a subject before the same target part of the subject is CT-scanned for further diagnosis, the X-ray two-dimensional radiographic image can be utilized as a scout image. Accordingly, the amount of X-ray irradiation of the subject (patient) as well as the amount of contrast media to be administered to the patient can be reduced.

In a fifth aspect, the X-ray hybrid diagnosis system consistent with the present invention further includes a mark member that is placed in a position relative to the subject. The X-ray two-dimensional radiographic image is obtained in the X-ray radiography unit for the subject of which a target part is determined relative to the mark member, and the projection data are acquired in the X-ray CT unit for the subject of which a target range is determined relative to the mark member. In the X-ray hybrid diagnosis system according to the fifth aspect of the present invention, the position of the mark member relative to the subject can be checked when the X-ray two-dimensional radiographic image is obtained or when the CT tomography image is obtained, and thus the position of the subject can be rendered recognizable during X-raying operations.

In a sixth aspect of the present invention, premised upon the aforementioned exemplary embodiments described as second and third aspects where the position information of the subject located in the X-ray CT unit is shown in the X-ray two-dimensional radiographic image, the position information shown in the X-ray two-dimensional radiographic image may includes a range of the subject for which the projection data are acquired in the X-ray CT unit. In the X-ray hybrid diagnosis system according to the sixth aspect of the present invention, the correspondence in position between the X-ray two-dimensional radiographic image and the tomography image is established and the range of the subject that has been CT-scanned can be indicated in the X-ray two-dimensional radiographic image. Therefore, an operator can easily recognize in the X-ray two-dimensional radiographic image the range of the subject that has been CT-scanned, and/or can easily recognize the correspondence in position between the X-ray two-dimensional radiographic image and the tomography image.

In a seventh aspect, the X-ray two-dimensional radiographic image obtained by the X-ray hybrid diagnosis system consistent with the present invention includes a first X-ray two-dimensional radiographic image and a second X-ray two-dimensional radiographic image that is different from the first X-ray two-dimensional radiographic image. A first tomography image corresponding to the first X-ray two-dimensional radiographic image and a second tomography image corresponding to the second two-dimensional radiographic image are displayed while the first X-ray two-dimensional radiographic image and the second X-ray two-dimensional radiographic image are displayed. In the X-ray hybrid diagnosis system according to the seventh aspect, even when two or more X-ray two-dimensional radiographic images are available, the correspondences in position between the X-ray two-dimensional radiographic images and tomography images corresponding thereto can be recognized.

In an eighth aspect, the display unit (as described above in conjunction with the second and third aspects, and particularly in conjunction with the sixth aspect) of the X-ray hybrid diagnosis system consistent with the present invention includes a pointer for indicating a position in the X-ray two-dimensional radiographic image to display a specific tomography image corresponding to the indicated position of the X-ray two-dimensional radiographic image. In the X-ray hybrid diagnosis system according to the eighth aspect of the present invention, when a specific position in the X-ray two-dimensional radiographic image is indicated with the pointer, the specific tomography image corresponding to the position in the X-ray two-dimensional radiographic image indicated with the pointer is displayed, because the correspondence in position between the X-ray two-dimensional radiographic image and the tomography image is established. Therefore, an operator can easily make a diagnosis of the tomography image corresponding to the part that the operator considers to be inspected in view of the X-ray two-dimensional radiographic image.

In a ninth aspect, the position information of the subject located in the X-ray radiography unit and the X-ray CT unit, as used in the X-ray hybrid diagnosis system consistent with the present invention, includes information on a position along a body axis of the subject. In the X-ray hybrid diagnosis system according to the ninth aspect of the present invention, its cradle may be oriented vertically in an upright position when X-ray two-dimensional radiographic image is obtained, but even in such a situation, the position can be managed easily because the position information determined with respect to a predetermined direction includes information on a position along a body axis of the subject.

According to the X-ray hybrid diagnosis system consistent with the present invention, position information for the X-ray CT scan operation can be associated with the X-ray two-dimensional radiographic image obtained in the X-ray radiography unit, and thus the correspondence in position between the X-ray two-dimensional radiographic image and the tomography image can be made recognizable. Therefore, in one embodiment, the X-ray two-dimensional radiographic image can be utilized as a scout image during the X-ray CT scan operation. In another embodiment where the tomography image obtained by scanning in the X-ray CT unit and the X-ray two-dimensional radiographic image obtained by the X-ray radiography unit are displayed concurrently, the correspondence in position between the images may be rendered easily recognizable.

### BRIEF DESCRIPTION OF THE DRAWINGS

The above-exemplified objects and aspects, other advantages and further features of the present invention will become readily apparent from the following description of illustrative, non-limiting embodiments with reference to accompanying drawings, in which:

FIG. 1 is a perspective view showing a setup of an X-ray hybrid diagnosis system 100;

FIG. 2 is a block diagram representing the X-ray hybrid diagnosis system 100;

FIG. 3 is a perspective view showing a setup of a CR unit 103;

FIGS. 4A, 4B and 4C are views showing a cradle 117 having a flat panel detector 70 incorporated therein;

FIG. 5 is a flowchart showing X-raying and diagnosis operations performed with the X-ray hybrid diagnosis system 100.

FIG. 6 is a flowchart showing CR and CT scan operations performed in turn;

FIGS. 7A, 7B, 7C, and 7D illustrate positioning of a patient (subject) in a case where X-ray two-dimensional radiographic images of the subject in a standing position are obtained by a CR operation and tomography images of the same subject are obtained through a CT scan operation;

FIGS. 8A and 8B are illustrations for explaining positional correspondences in a Z-axis direction between the two-dimensional radiographic image obtained by the CR operation and the tomography image obtained through a CT scan operation;

FIGS. 9A, 9B, and 9C illustrate an operation of specifying a range in which tomography images are to be obtained through a CT scan operation, using a two-dimensional radiographic image obtained by the CR operation;

FIG. 10 shows an exemplary display representation which contains multiple two-dimensional radiographic images obtained by the CR operation and multiple tomography images obtained for CT scan ranges specified in respective two-dimensional radiographic images; and

FIG. 11 shows a specific example of the display representation which contains one two-dimensional radiographic image obtained by the CR operation and multiple tomography images obtained for a CT scan range specified in the two-dimensional radiographic image.

### DETAILED DESCRIPTION OF THE EXEMPLARY EMBODIMENTS

<General Arrangement of X-Ray Hybrid Diagnosis System>

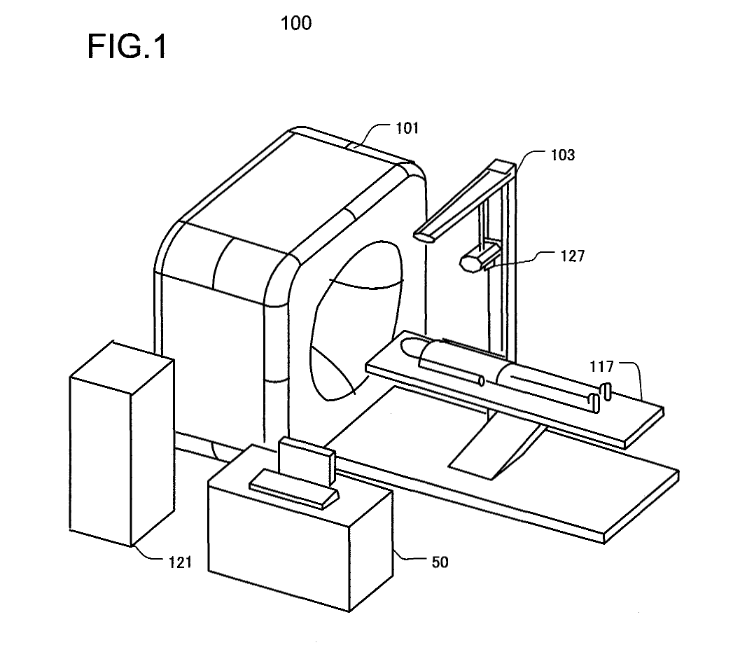

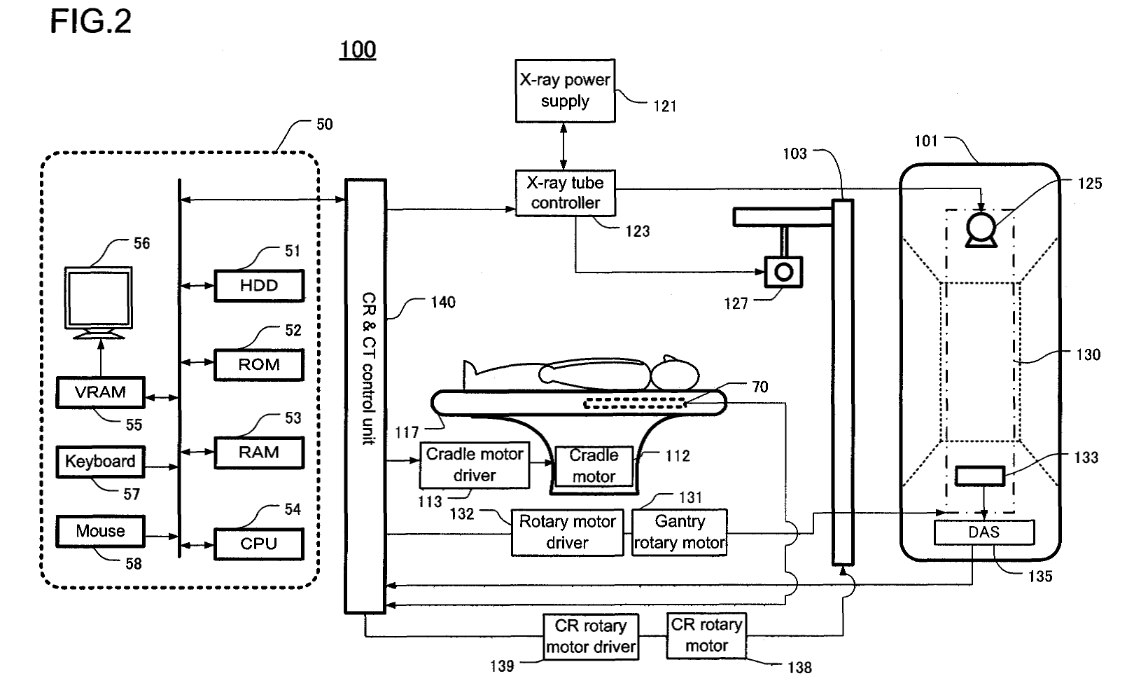

FIG. 1 is a perspective view showing a general arrangement of an X-ray hybrid diagnosis system 100 according to a first exemplary embodiment of the present invention. FIG. 2 is a block diagram representing an arrangement of the X-ray hybrid diagnosis system 100 according to one exemplified embodiment of the present invention. This system generally includes an operation console 50, a gantry 101, an X-ray power supply 121, and a CR unit 103. The gantry 101 is a computed tomography or CT unit adapted to acquire X-ray projection data to obtain tomography images of an examinee's body. The CR unit 103 is a computed radiography unit (digital X-ray imager) adapted to obtain X-ray radiographic images of the examinee's body. The operation console 50 is adapted to reconstruct an X-ray tomography image of an examinee's body based upon data transmitted from the gantry 101 and to display the X-ray tomography image. The operation console 50 is also adapted to display an X-ray radiographic image based upon data transmitted from a flat panel detector (denoted by 70 in FIG. 2).

Not every component of the X-ray hybrid diagnosis system 100 need be placed in one and the same room. For example, the gantry 101 and the CR unit 103 may be placed in a consulting room in which patients as examinees are diagnosed, whereas the operation console 50 may be placed in an operation room for a radiographer. The X-ray power supply 121 for powering the X-ray tube 125 in the gantry 101 and the X-ray tube 127 in the CR unit 103 may be placed in a basement in order to free up a space in the consulting room or operation room.

The cradle 117 is movable, with a subject laid thereon in a decubitus position; The CR unit 103 is disposed at one side of the cradle 117.

The gantry 101 and the CR unit 103 are communicatively coupled with a CR & CT control unit 140 and various other devices which will be described later, and are configured to operate under control of the CR & CT control unit 140.

Inside the gantry 101 are provided an X-ray tube 125 for producing X rays, an X-ray tube controller 123 connected with the X-ray tube 125, a collimator (not shown) for limiting a range of irradiation of X rays, a control motor (not shown) connected with the collimator for regulating a dimension of an opening (slit or aperture) (not shown) of the collimator, and a collimator driver (not shown). X rays that have passed through the collimator form a fan-shaped beam (so-called “fan beam”) of X rays.

[Also provided inside the gantry 101 is an X-ray detection unit 133, which includes multiple rows of detection channels each having a plurality of detectors. Each detector has a length depending upon a fan angle (normally 60□ or so). The detection channels are arranged in a direction (element direction) along the Z-axis direction. The X-ray detection unit 133 is, for example, made up of a scintillator and a photodiode used in combination.

The gantry 101 includes at least one data acquisition unit or DAS (standing for Data Acquisition System) 135 which acquires projection data from outputs of the detection channels. The number of the data acquisition unit(s) 135 may be one or more (e.g., four, eight, sixteen or thirty two), and each data acquisition unit 135 is connected with the X-ray detection unit 133. For example, the gantry 101 including four data acquisition units 135, which is normally called “4DAS”, includes the detection channels arranged in four rows in the element direction, and can obtain four slice images in one cycle of revolution of the X-ray tube 125. The X-ray tube 125 and the X-ray detection unit 133 are disposed in opposite positions in the gantry 101 such that a hollow space for accommodating a subject is left between the X-ray tube 125 and the X-ray detection unit 133. The X-ray tube 125 and the X-ray detection unit 133 are attached to a gantry rotor 130 so that the X-ray tube 125 and the X-ray detection unit 133 revolve around the subject while maintaining the opposed positions relative to each other. A gantry rotary motor 131 and a gantry rotary motor driver 132 are connected with the gantry rotor 130, and the gantry rotor 130 is regulated by the gantry rotary motor driver 132 to make one rotation in about 0.3 second to about 1.0 second.

The X-ray hybrid diagnosis system 100 provides user-selectable options of operation modes: a full-scan mode in which images are reconstructed from projection data of 360° and a half-scan mode in which images are reconstructed from projection data of 180° plus one unit fan angle. Each scan mode offers its own peculiar advantage: high-quality tomography images can be reconstructed in the full-scan mode, while increased scanning speed, which can be obtained at the expense of some resolution of the tomography images, in the half-scan mode leads to reduction in exposure of a subject to radiation.

The CR unit 103 includes an X-ray tube 127 for producing X rays and a the collimator (not shown) having an opening for limiting a range of irradiation of X rays produced in the X-ray tube 127. The X-ray tube controller 123 is connected with the X-ray tube 127. Also provided in the cradle 117 is a flat panel detector 70 adapted to receive X-rays from the X-ray tube 127.

The position of the X-ray tube 127 and the flat panel detector 70 can be adjusted through six degrees of freedom, in accordance with the posture (standing, sitting or decubitus position) of the subject or the portion to be radiographed of the subject. For that purpose, a CR rotary motor 138 and a CR rotary motor driver 139 are connected with the CR unit 103.

The cradle 117 is moved in the body-axial direction of the subject (i.e., Z-axis direction) by a cradle motor 112. The cradle motor 112 is actuated by a cradle motor driver 113.

Additionally, an electrocardiograph for transducing a heartbeat into an electric signal may be attached if necessary to the subject in order to check the heartbeat conditions of the subject. By providing the signal from the electrocardiograph to the CT & CR control unit 140, irradiation of X rays can be carried out in accordance with the heartbeat conditions of the subject.

The CT & CR control unit 140 is communicatively coupled with the operation console 50. Responsive to instructions from the operation console 50, various control signals are transmitted to the X-ray tube controller 123, the cradle motor driver 113, the rotary motor driver 132 and the like. Data acquired by the data acquisition unit 135 are transmitted to the operation console 50 in which images are reconstructed and tomography images are displayed. Similarly, data obtained by the flat panel detector 70 are transmitted to the operation console 50 in which two-dimensional radiographic images are displayed.

The operation console 50 is typically embodied in a workstation, as illustrated in FIG. 2, which mainly includes a ROM 52 storing a boot program and the like, a RAM 53 serving as a main memory and a CPU 54 executing instructions for controlling the entire system.

A hard disk drive or HDD 51 is provided in the operation console 50 to store not only an operating system but also image-processing programs for providing various instructions given to the gantry 101 and the CR unit 103 and instructions to display two-dimensional radiographic images based upon data received from the flat panel detector 70, as well as image-processing programs for reconstructing and displaying X-ray tomography images based upon data received from the data acquisition unit 135. A VRAM 55 is a memory in which image data to be displayed are deployed, that is, the image data, etc. can be deployed in the VRAM 55 and thereby displayed in a monitor 56. Operators use a keyboard 57 and a mouse 58 to perform a variety of operations and manipulations.

<CR Unit 103 Setup>

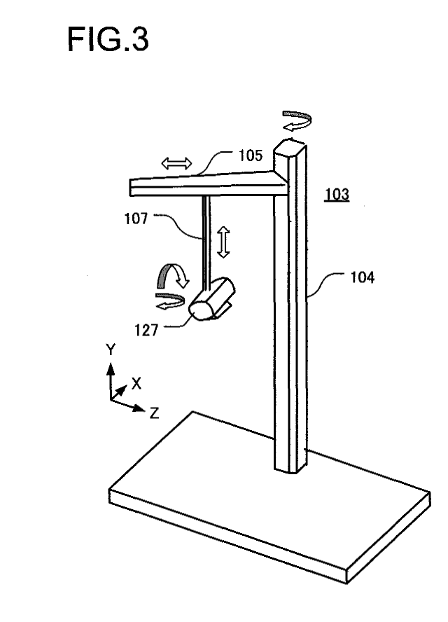

FIG. 3 is a perspective view showing a setup of a CR unit 103. A frame of the CR unit 103 is comprised of a rotation support post 104, a swivel arm 105 provided at an upper portion of the rotation support post 104, and an extendable arm 107 suspended from the swivel arm 105. The X-ray tube 127 is provided at an end of the extendable arm 107 in a manner that allows the X-ray tube 127 to rotate via a ball joint mechanism.

<Cradle Setup>

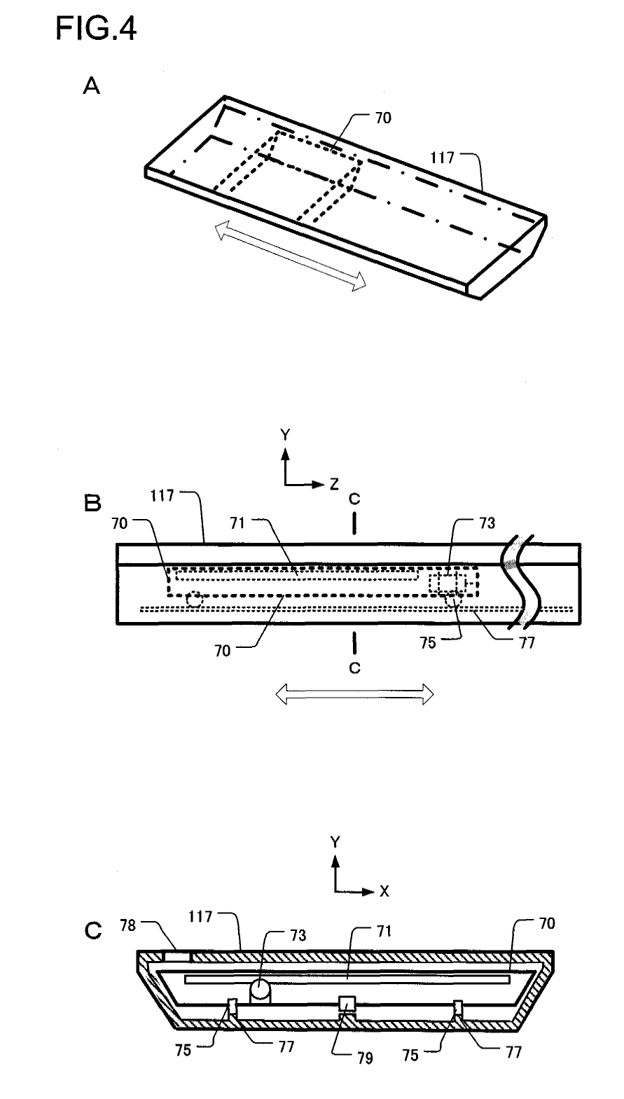

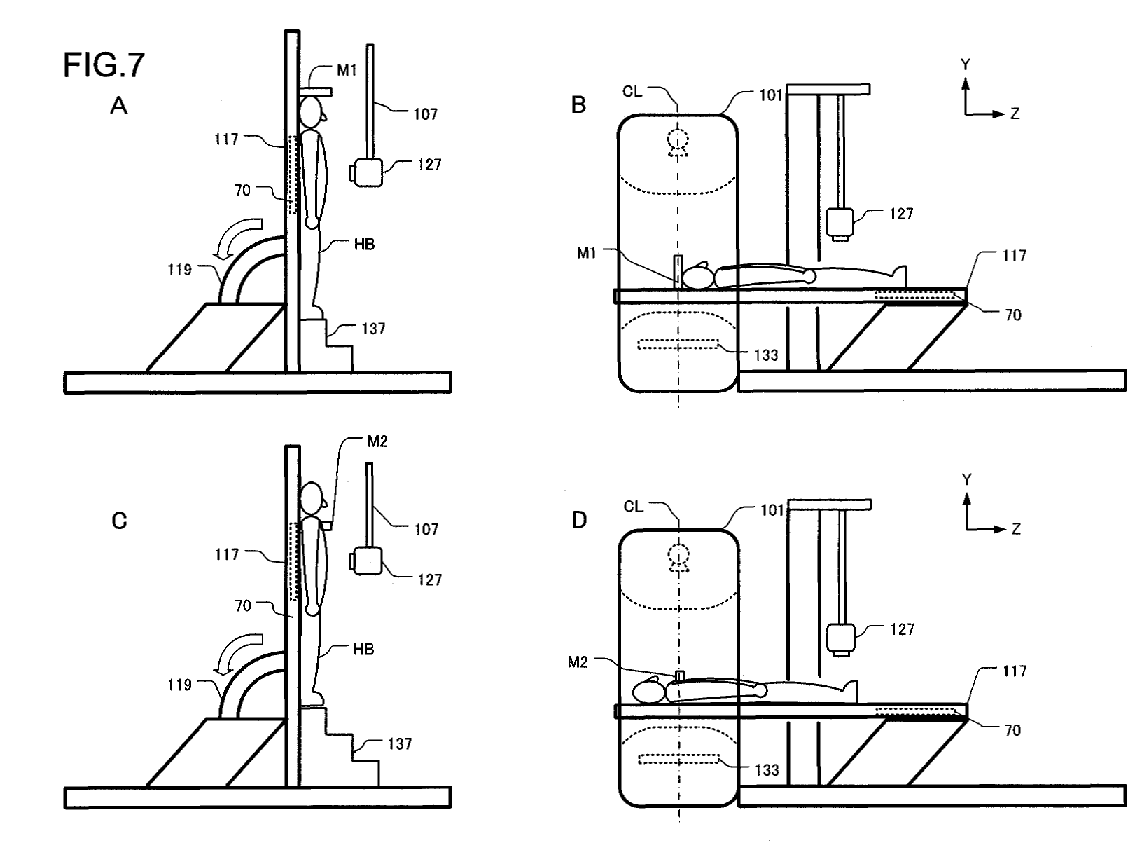

FIGS. 4A through 4C show a structure of a cradle 117. FIG. 4A is a perspective view of the cradle 117, FIG. 4B is a phantom showing the cradle 117 in cross section, and FIG. 4C is a tomography view taken along line C-C of FIG. 4B. As shown in FIG. 4A, the cradle 117 has a hollow space and made of X-ray transparent material such as plastic. In this hollow space is provided a flat panel detector 70 that is movable bidirectionally along the Z axis as indicated by an arrow. The cradle 117 can move in the Z-axis directions on a table, and can be raised upright by a raising drive unit 119 comprised of an air cylinder or the like, as will be described with reference to FIGS. 7A and 7C.

As shown in FIGS. 4B and 4C, guide rails 77 are provided in the hollow space of the cradle 117 so that the flat panel detector 70 can smoothly move in a specific direction. The guide rails 77 are made of X-ray transparent hard plastic or the like so that the guide rails 77 do not cast the shadow on X-ray CT scan images. The length of the guide rails 77 is equal to the length of the cradle 117 in the Z-axis direction. Four tires 75 corresponding to the guide rails 77 are provided on the flat panel detector 70. A driving motor 73 is provided in the flat panel detector 70 to drive the tires 75. A two-dimensional panel sensor 71 is provided on an X-Z plane in the flat panel detector 70. The two-dimensional panel sensor 71 is comprised for example of a scintillator and a sensor, such as CCD sensor, MOS sensor, or CMOS sensor. When the X-ray CT scan is performed, the flat panel detector 70 has been moved to a retracted position that is at the end of the cradle 117 facing toward the +Z-axis direction. Therefore, the two-dimensional panel sensor 71, driving motor 73 and tires 75 incorporated in the flat panel detector 70 may contain materials, such as metal, which are not completely transparent to X rays, without any problem.

A transparent window 78 made of plastic is formed in a part of a top plate of the cradle 117. This allows an operator to visually check where the flat panel detector 70 is located in actuality. The transparent window 78 may preferably be provided near a side of the top plate of the cradle 117 so that the position of the flat panel detector 70 can be checked even when a patient is laid on the cradle 117 in a decubitus position. A center line is marked on the top face of the flat panel detector 70 so that the center of the two-dimensional panel sensor 71 along the length in the Z-axis direction can be seen through the transparent window 78.

In order to supply power to the two-dimensional panel sensor 71 and the driving motor 73, a power cable (not shown) is provided between the flat panel detector 70 and the cradle 117, and likewise a signal line through which a signal is output from the two-dimensional panel sensor 71 is provided between the flat panel detector 70 and the cradle 117. As shown in FIGS. 4B and 4C, the driving motor 73 is arranged in the flat panel detector 70 in this embodiment, but may alternatively be arranged in the cradle 117. Further provided in the cradle 117 is, as shown in FIG. 4C, a position sensor 79 for detecting where (in the Z-axis direction) in the cradle 117 the flat panel detector 70 is located. In an embodiment where the driving motor 73 is a stepping motor or the like, the position of the flat panel detector 70 can be detected if the position of the flat panel detector 70 is initialized every time upon startup, and thus such a position sensor 79 would not necessarily required.

<X-Raying Operation Using X-ray Hybrid Diagnosis System>

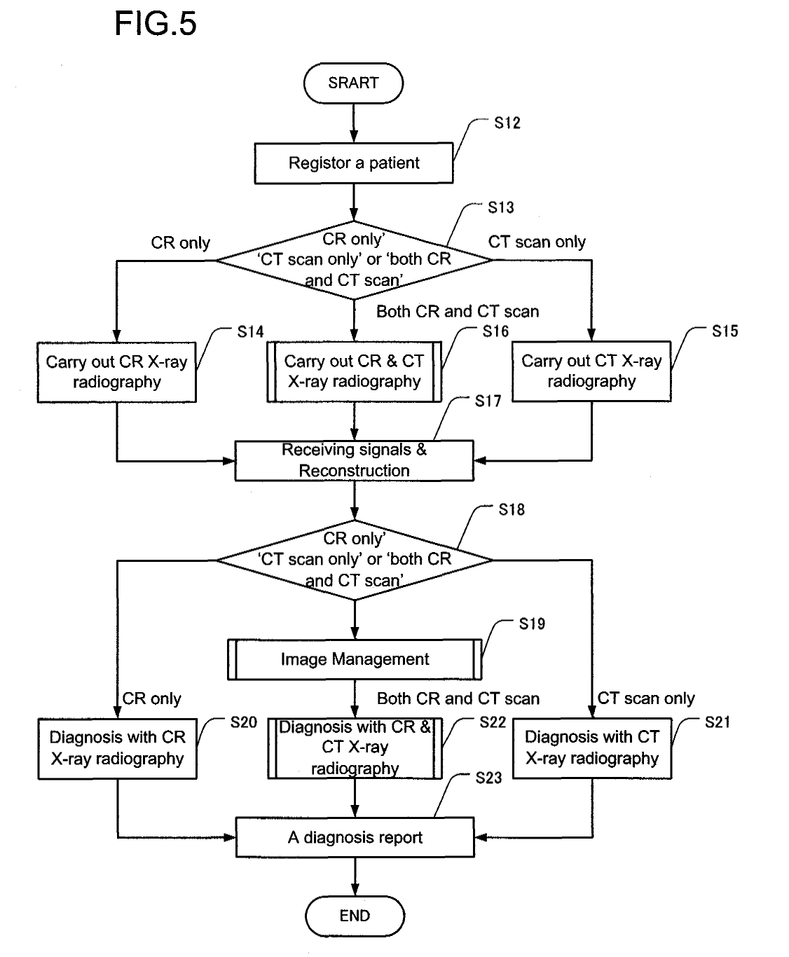

FIG. 5 is a flowchart showing exemplary X-raying, imaging and diagnosis operations using the X-ray hybrid diagnosis system 100. X-raying operations in the X-ray hybrid diagnosis system 100 may be performed in one of several (generally four) scan types provided as options. The scan types include: TYPE 1 in which only CR is performed (CR mode); TYPE 2 in which only CT scan is performed (CT scan mode); TYPE 3 in which CR is followed by CT scan; and TYPE 4 in which CT scan is followed by CR.

In step S12, an entry of a patient is made at the operation console 50. A desirable scan type that is determined in view of the part to be X-rayed and symptoms of the patient is input to specify which is to be carried out, ‘CR only’ or ‘CT scan only’ or ‘both CR and CT scan’. In step S13, a determination is made as to which has been specified, ‘CR only’ or ‘CT scan only’ or ‘both CR and CT scan’, based upon the input scan type.

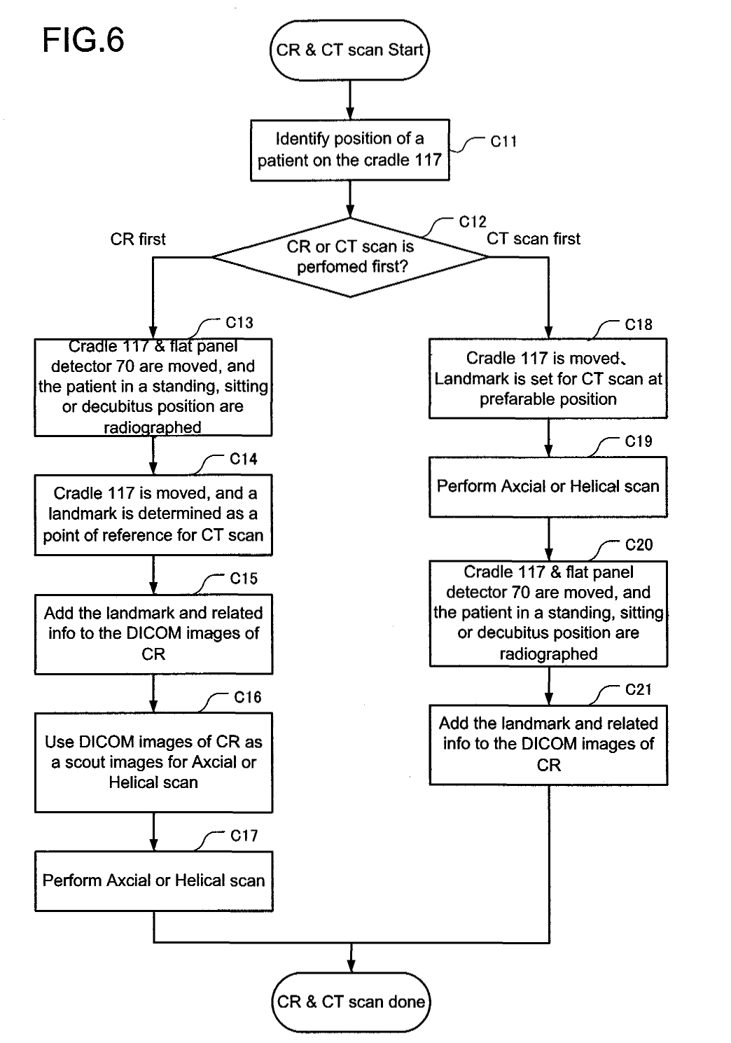

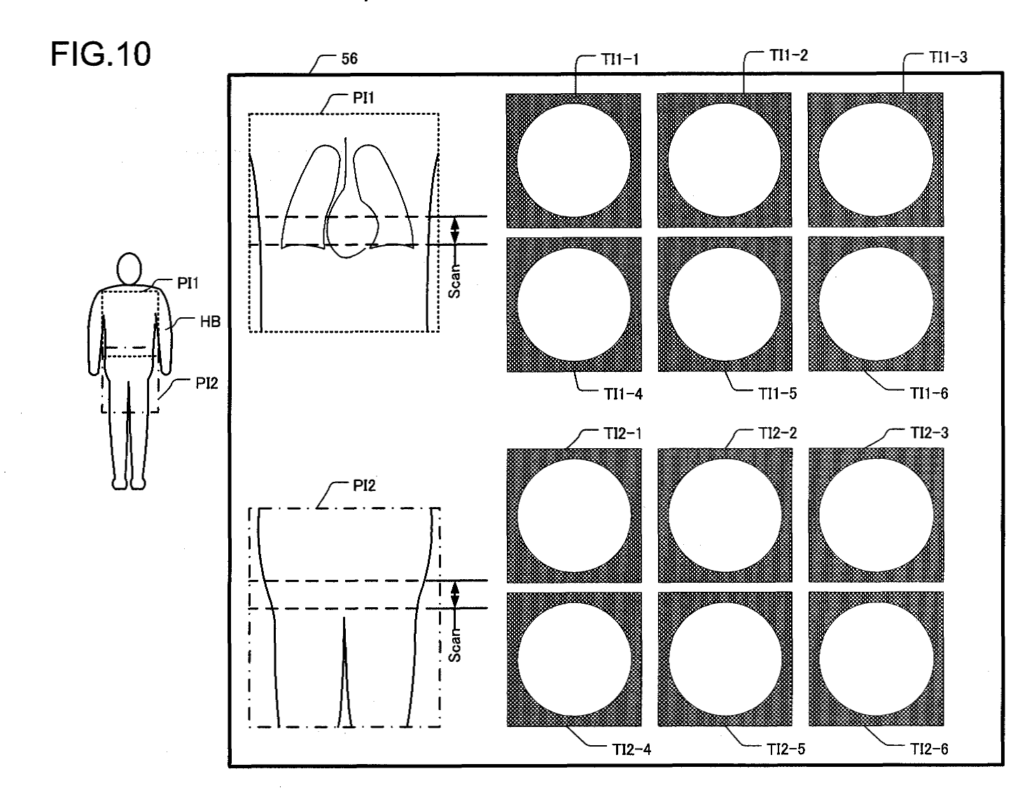

If it is determined that ‘CR only’ has been specified, then the process goes to step S14 in which the X-ray tube 127 and the flat panel detector 70 in the CR unit 103 are moved in accordance with the part to be X-rayed. X-ray radiography is then carried out. If it is determined that ‘CT scan only’ has been specified, then the process goes to step S15 in which the X-ray tube 125 and the cradle 117 in the CT unit (gantry) 101 are moved in accordance with the part to be X-rayed. CT scan is then carried out. If it is determined that ‘both CR and CT scan’ has been specified, then the process goes to step S16 in which the X-ray tube 127 and the flat panel detector 70 in the CR unit 103 are moved and the X-ray tube 125 and the cradle 117 in the CT unit (gantry) 101 are moved in accordance with the part to be X-rayed. Specific X-raying operations in this instance will be described later with reference to FIG. 6 and FIG. 10.

In step S17, irrespective of the implemented scan type, the operation console 50 receives signals from the flat panel detector 70 and/or the X-ray detection unit 133 through the CR & CT control unit 140, and performs necessary image processing, which includes for example reconstruction of images and other operations, to obtain two-dimensional radiographic images and/or tomography images. In step S18, it is determined which scan type has been implemented, ‘CR only’ or ‘CT scan only’ or ‘both CR and CT scan’.

If it is determined that the scan type is ‘CR only’, then the process goes to step S20 in which the X-ray two-dimensional radiographic images obtained in the CR unit 103 are displayed on the monitor 56. The operator conducts a diagnosis using the obtained X-ray two-dimensional radiographic images. If it is determined that the scan type is ‘CT scan only’, then the process goes to step S21 in which the tomography images obtained by scanning in the CT unit (gantry) 101 are displayed on the monitor 56. The operator conducts a diagnosis using the CT-scan tomography images, etc. If it is determined that the scan type is ‘both CR and CT scan’, then the process goes to step S19 in which the X-ray two-dimensional radiographic images obtained in the CR unit 103 and the tomography images obtained by scanning in the CT unit (gantry) 101 are subjected to image management. What and how the image management is carried out may for example be specified by the operator in advance. The image management in this step is the management relating to display modes, which determine for example how many X-ray two-dimensional radiographic images and CT-scan tomography images are displayed on the monitor 56.

Subsequent to step S19, the process proceeds to step S22 in which the X-ray two-dimensional radiographic images obtained in the CR unit 103 and the CT-scan tomography images obtained by scanning in the CT unit (gantry) 101 are displayed on the monitor 56. The operator conducts a diagnosis while viewing the X-ray two-dimensional radiographic images and the CT-scan tomography images. Next, subsequent to step S20, S21 or S22, the operator compiles a diagnosis report at the operation console 50 in step S23.

<CR & CT Scan>

A detailed description of the CR and CT scan operations as mentioned above in step S16 of FIG. 5 is given with reference to the flowchart shown in FIG. 6.

Referring now to FIG. 6, the position of a subject (patient) on the cradle 117 is located in step C11. This is because it is not clear where on the cradle 117 the patient is positioned. When the patient is to be radiographed with X rays in the CR unit 103, the patient comes in a standing, sitting or decubitus position and gets X-rayed. When the patient is lying on the cradle 117 in a decubitus position, the position of the patient would not get deviated so much at the time of shifting from X-ray radiography operation in the CR unit 103 to CT scan operation in the CT unit (gantry) 101 or from CT scan operation in the CT unit (gantry) 101 to X-ray radiography operation in the CR unit 103. However, when the patient is in a standing or sitting position during the X-ray radiography operation in the CR unit 103, the position of the patient would possibly get deviated. Therefore, it is preferred that the position of the subject on the cradle 117 be located as in step C11.

Turning to FIGS. 7A to 7D, the above-mentioned step C11 is described in more detail. In FIGS. 7A and 7C, the patient (subject) is in a standing position for radiography carried out in the CR unit 103 to obtain X-ray two-dimensional radiographic images. FIGS. 7B and 7D show states, shifted from the states of FIGS. 7A and 7C, respectively, for CT scan carried out in the CT unit (gantry) 101 to obtain tomography images. In FIG. 7A, a chest of the patient in a standing position is radiographed by the CR unit 103 to obtain X-ray two-dimensional radiographic images. In response to an instruction from the operation console 50, the X-ray tube 127 and the flat panel detector 70 are arranged into specific positions. The cradle 117 is raised upright by a raising drive unit 119 comprised of an air cylinder or the like. Stairs 137 may be used to allow the patient to stand against the cradle 117 as necessary. In FIG. 7A, the cradle 117 is provided with a mark M1. The patient lies on the cradle 117 with the vertex of his/her head brought in contact with the mark M1. This makes it possible to determine where on the cradle 117 the patient is positioned. As shown in FIG. 7B, the cradle 117 arranged in a horizontal position is moved into the hollow space in the gantry 101 so as to carry out CT scan for obtaining tomography images. In this operation, a center line CL produced between the center of the X-ray tube 125 and the center of the X-ray detection unit 133 in the Z-axis direction is aligned with the mark M1. In this way, the position of the patient on the cradle 117 is located by using the mark M1 as a point of reference.

In FIG. 7C as well, the chest of the patient in a standing position is radiographed by the CR unit to obtain X-ray two-dimensional radiographic images. In response to an instruction from the operation console 50, the X-ray tube 127 and the flat panel detector 70 are arranged into specific positions. The cradle 117 is raised upright by the raising drive unit 119 comprised of an air cylinder or the like. In FIG. 7C, a mark M2 is provided on a breast of a garment which the patient wears. This makes it possible to determine where on the cradle 117 the patient is positioned. As shown in FIG. 7D, the cradle 117 arranged in a horizontal position is moved into the hollow space in the gantry 101 so as to carry out CT scan for obtaining tomography images. In this operation, a center line CL produced between the center of the X-ray tube 125 and the center of the X-ray detection unit 133 in the Z-axis direction is aligned with the mark M2. In this way, the position of the patient on the cradle 117 is located by using the mark M2 as a point of reference.

The position in the gantry 101 to be aligned with the mark M1 or M2 may not necessarily be the center line CL. For example, the entrance or exit of the gantry 101 or other part fixed relative to the gantry 101 may be used as the position for alignment. Alternatively, a light-emitting position of the positioning light, such as a halogen lamp, a laser or the like, for use in positioning and checking a slicing position of a subject may be used for alignment. The following discussion is, however, based on the premise that the center line CL is adopted as a standard position for alignment. When the patient is in a sitting position, the mark M2 may be provided on the patient as shown in FIGS. 7C and 7D. The point of reference can be recognizable only if the patient wears a garment, because the mark M2 is attached to the garment which the patient wears. When the X-ray CT scan is performed, the flat panel detector 70 has been moved to a retracted position that is at the end of the cradle 117 facing toward the +Z-axis direction.

Anything that is X-ray transparent and visually recognizable can be used as the marks M1 and M2. For example, colored plastic tape, or the like is applicable. In an embodiment where the mark M1, M2 is recognized with a reflection sensor instead of unaided eye, plastic tape the surface of which is coated with reflective film may be used.

To provide a common set of coordinates, the coordinates for use in CT scan operation may be predefined with consideration given to such instances that the legs of the subject are oriented toward the +Z-axis direction or the head of the subject is oriented toward the +Z-axis direction, for example. Similarly, the coordinates for use in CR operation may be predefined with consideration given to such instances that the subject is in a standing, sitting or decubitus (in which case the legs may be oriented toward the +Z-axis direction or the head may be oriented toward the +Z-axis direction) position. With this in view, for example, the position of the body axis (longitudinal axis) of the subject laid in a decubitus position may be defined as the Z axis in the system 100, and the orientation of the head of the subject may be defined as the −Z-axis direction, so that the positions of each component of the system 100 may be converted into those plotted in a common coordinate system.

Referring back to FIG. 6, in step C12, it is determined from the instruction input at the operation console 50 which is performed first, CR by the CR unit 103 or CT scan by the CT unit 101. If it is determined that the CR is performed first, the process goes to step C13, while if it is determined that the CT scan is performed first, the process goes to step C18.

In step C13, the cradle 117 and the flat panel detector 70 are moved, and target parts of the patient in a standing, sitting or decubitus position are radiographed by the CR unit 103. The position of the flat panel detector 70 in the cradle 117 can be detected, as described above with reference to FIG. 4C, with the position sensor 79, for example. The position of the patient relative to the cradle 117 can be determined, as described above with reference to FIGS. 7A through 7D, with the mark M1 or M2, for example. The X-ray two-dimensional radiographic images may be saved in any format, and it is preferred that the images be handled in a format compliant with the standard for DICOM (digital imaging and communication in medicine).

Next, in step C14, the cradle 117 is moved for CT scan, and a landmark is determined as a point of reference for CT scan operation in a position preferable to the CT scan. Then, in step C15, the landmark and relevant position information are added to the X-ray two-dimensional radiographic images obtained by the CR operation. By adding the landmark for CT scan operation to the X-ray two-dimensional radiographic images, the correspondence in position is established between the CT scan by the CT unit (gantry) 101 and the X-ray radiography by the CR unit 103.

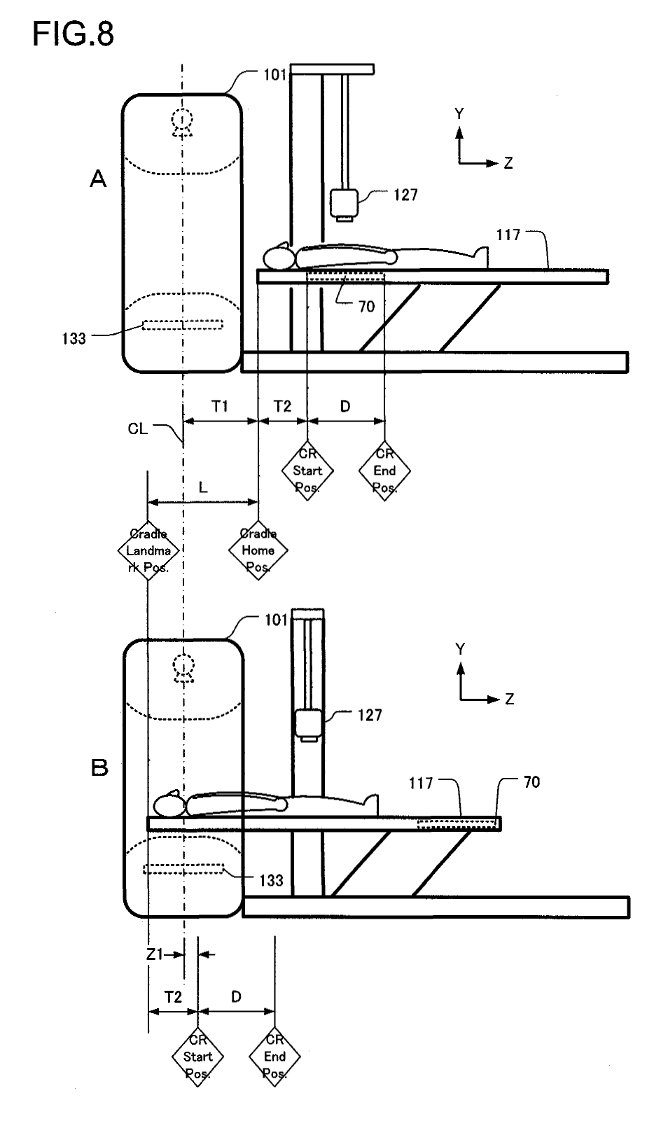

Turning to FIGS. 8A and 8B, the correspondence in position between the CT scan and the X-ray radiography are described in more detail. FIG. 8A illustrates the X-ray radiography carried out by the CR unit 103 for the patient in a decubitus position. FIG. 8B illustrates the CT scan carried out by the CT unit 101. Reference characters used in FIGS. 8A and 8B are as follows:

T1: Distance (fixed value) between center line CL of gantry 101 and a front end of cradle 117 during CR operation;

T2: Distance (variable value) between the front end of cradle 117 and a front end of flat panel detector 70 during CR operation;

D: Length (fixed value) of flat panel detector 70, i.e., distance between the front and rear ends of flat panel detector 70; and

L: Distance (variable value) between a position in which the landmark is set and an initial position of cradle 117, i.e., a landmark value.

X-ray radiography is carried out by the CR unit 103 in the state as shown in FIG. 8A. During CR operation, the cradle 117 is in the initial position. The flat panel detector 70 is moved to a position corresponding to a part of the subject to be X-rayed. In FIG. 8A is shown a state in which a chest of the subject is radiographed. The position of the flat panel detector 70 can be detected with the position sensor 79 as described above with reference to FIG. 4C. Accordingly, the distance T2 can be determined. Then, the CT scan operation is carried out. In FIG. 8B, the operator sets a landmark at the front end of the cradle 117 when the center line CL of the gantry 101 is positioned on the neck of the subject. The flat panel detector 70 is moved to the right edge (an end facing toward the +Z-axis direction) of the cradle 117 so as not to interfere with the CT scan operation.

When the subject is oriented and enters the gantry 101 from its head, start and end positions of radiography carried out by the CR unit 103 are related to the position of the landmark as follows:

CR_Start_Position=L-T1-T2;

CR_End_Position=L-T1-T2-D.

When the subject is oriented and enters the gantry 101 from its legs, the start and end positions of radiography carried out by the CR unit 103 are related to the position of the landmark as follows:

CR_Start_Position=-(L-T1-T2);

CR_End_Position=-(L-T1-T2-D).

The start and end positions of radiography carried out by the CR unit 103 may have a positive value or a negative value. Therefore, values resulting from the above equations may be absolute values if a sign S is prefixed to a positive value and a sign I is prefixed to a negative value. In FIG. 8B, to be more specific, the following equation is satisfied: CR_Start_Position=L-T1-T2=−Z1, and the relation between the position of the landmark and the start position of radiography carried out by the CR unit 103 may be expressed in I|Z1|. In this way, correspondence can be established between the start and end positions of radiography carried out by the CR unit 103 and the position of the landmark, so that correspondence in position can be established between the X-ray two-dimensional radiographic image obtained by the CR operation and the CT scan tomography image.

Returning to FIG. 6 again, in step C16, the X-ray two-dimensional radiographic image obtained by the CR operation is used for positioning in the CT scan operation. To compare step C16 with step S15 of FIG. 5, it is noted that only a CT scan operation is performed in step S15, where the cradle 117 is moved while the rotor 130 remains immovable so as to obtain a scout image which is used merely for the positioning in the CT scan operation.

In contrast, the operation in step C16 utilizes the X-ray two-dimensional radiographic image with landmark information incorporated therein obtained in step C15 as a scout image. Since the landmark in CT scan operation and the landmark in the X-ray two-dimensional radiographic image coincide with each other, the operator can specify a range to be CT-scanned while viewing the X-ray two-dimensional radiographic image displayed on the monitor 56.

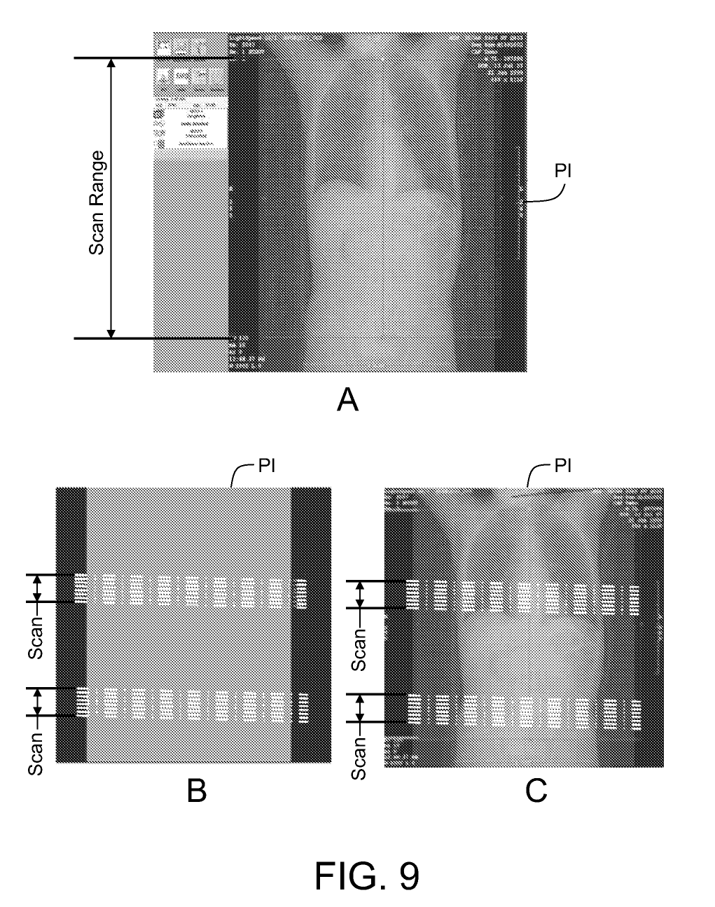

More specifically, images as shown in FIGS. 9A to 9C may be displayed on the monitor 56. FIG. 9A is a radiograph for specifying a target range for tomography image T1 to be obtained by the subsequent CT scan operation by utilizing the X-ray two-dimensional radiographic image PI obtained by the CR operation. FIG. 9B is a radiograph in which a target range to be CT-scanned is shown. FIG. 9C is a radiograph in which the target range to be CT-scanned is overlaid on the X-ray two-dimensional radiographic image PI obtained by the CR operation. It is shown in FIGS. 9B and 9C that two different scan ranges for tomography images are specified in the same X-ray two-dimensional radiographic image PI obtained by a single CR operation.

Returning to FIG. 6 again, the scan range specified in step C16 is CT-scanned in step C17.

If it is determined in step C12 that the CT scan is performed first, the process goes to step C18 in which the cradle 117 is moved to a position preferable to the CT scan operation, and a landmark as a point of reference for the CT scan operation is determined. The cradle 117 is then moved while the rotor 130 remains immovable so as to obtain a scout image.

In step C19, a scan range in which tomography images are to be obtained is specified based upon the scout image obtained by scanning in the CT unit 101, and the specified scan range to be CT-scanned is subjected to a conventional (axial) or helical scan operation.

In step C20, the cradle 117 and the flat panel detector 70 are moved and a necessary part of the subject in a standing, sitting or decubitus position is radiographed by the CR unit 103.

In step C21, a landmark and related position information are added to the X-ray two-dimensional radiographic image obtained by the CR unit 103. By adding the landmark for the CT scan operation to the X-ray two-dimensional radiographic image, the correspondence in position is established between the CT scan operation carried out by the CT unit (gantry) 101 and the CR operation carried out by the CR unit 103. When another CT scan operation is to be carried out, the same X-ray two-dimensional radiographic image can be used.

<Image Management>

The next discussion will focus on the operation performed in step S19 of FIG. 5 to associate the tomography images obtained by scanning in the CT unit 101 with the X-ray two-dimensional radiographic images obtained by the CR unit 103.

There are multiple X-ray two-dimensional radiographic images PI obtained by the CR unit 103, and each X-ray two-dimensional radiographic image PI has corresponding multiple tomography images TI to be obtained through scanning in the CT unit 101. In such an instance, the image management to be performed is, for example, as follows.

X-ray two-dimensional radiographic image PI1 obtained by CR operation:

CT scan tomography image TI1-1 (associated with image PI1);

CT scan tomography image TI1-2 (associated with image PI1); . . . ;

CT scan tomography image TI1-98 (associated with image PI1);

CT scan tomography image TI1-99 (associated with image PI1);

X-ray two-dimensional radiographic image P12 obtained by CR operation:

CT scan tomography image TI2-1 (associated with image PI2);

CT scan tomography image TI2-2 (associated with image PI2); . . . ;

CT scan tomography image TI2-55 (associated with image PI2);

CT scan tomography image TI2-56 (associated with image PI2);

X-ray two-dimensional radiographic image PI5 obtained by CR operation:

CT scan tomography image A01 (associated with image PI5);

CT scan tomography image A02 (associated with image PI5);

CT scan tomography image A19 (associated with image PI5);

CT scan tomography image B01 (associated with image PI5);

CT scan tomography image B02 (associated with image PI5); . . . ;

CT scan tomography image B29 (associated with image PI5),

where the X-ray two-dimensional radiographic image PI5 has CT scan range A and CT scan range B, and the tomography images for the both ranges A and B are associated with the X-ray two-dimensional radiographic image PI5.

FIG. 10 shows an exemplary display representation which contains multiple two-dimensional radiographic images PI obtained by the CR operation and multiple tomography images TI obtained for CT scan ranges specified in the two-dimensional radiographic images PI. In an example shown in FIG. 10, the X-ray two-dimensional radiographic images PI1 and the X-ray two-dimensional radiographic images PI2 are obtained by CR operations targeted at the chest and abdomen of the patient, respectively. Each of the X-ray two-dimensional radiographic images PI1 and PI2 is used as a scout image to determine a target range to be CT-scanned, and the tomography images T1 corresponding to the target range are obtained. The operator has specified in advance the number of X-ray two-dimensional radiographic images PI to be displayed on the monitor 56 and the number tomography images TI corresponding thereto to be displayed on the monitor 56 at the same time. FIG. 10 shows an example in which two X-ray two-dimensional radiographic images PI and six tomography images TI corresponding thereto are displayed in accordance with a layout as specified by the operator.

In the example shown in FIG. 10, X-ray two-dimensional radiographic images PI1 and PI2 are displayed respectively in upper and lower left areas on the monitor 56. In the X-ray two-dimensional radiographic images PI1 and PI2, there are shown the ranges in which tomography images have been obtained. In an area at the right of the X-ray two-dimensional radiographic image PI1, the first six tomography images TI1-1 to TI1-6 in the scan range of the X-ray two-dimensional radiographic image PI1 are displayed. Similarly, in an area at the right of the X-ray two-dimensional radiographic image PI2, the first six tomography images TI2-1 to TI2-6 in the scan range of the X-ray two-dimensional radiographic image PI2 are displayed. The tomography images to be displayed are not necessarily be the first six, and may be selected otherwise. For example, six tomography images selected one from each uniformly divided block of the scan range may be selected.

<Diagnosis Using X-Ray Two-Dimensional Radiographic Image and CT-Scan Cross-Sectional Image>

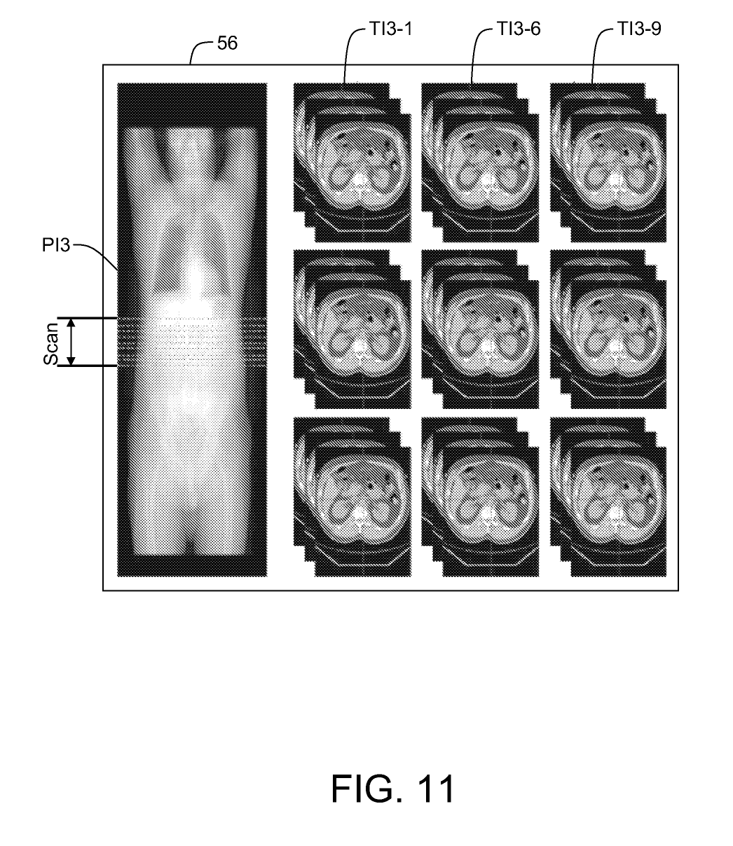

In step S22 of FIG. 5, the X-ray two-dimensional radiographic images obtained in the CR unit 103 and the CT-scan tomography images obtained by scanning in the CT unit (gantry) 101 are displayed on the monitor 56. FIG. 11 shows a specific example of the display representation which contains one two-dimensional radiographic image PI3 obtained by the CR operation and twenty-seven tomography images TI3 obtained for a CT scan range specified in the two-dimensional radiographic image PI3. The operator conducts a diagnosis while viewing the both of the X-ray two-dimensional radiographic image PI3 and the CT scan tomography images TI3 (TI3-1, TI3-2 . . . ). If the operator double-clicks on any one of the tomography images TI3 on the monitor 56 with a pointer operated by a mouse 58, the operator can see the tomography image TI3 under magnification. If the operator double-clicks on part of the scan range shown in the X-ray two-dimensional radiographic image PI3 with the pointer, the operator can see three tomography images TI3 located in the part of the scan range under magnification. The operator can roughly grasp a target part to be diagnosed with the X-ray two-dimensional radiographic image PI3, and can observe a tomography image TI3 corresponding to a specific part which the operator wishes to view more in detail by moving the pointer using the mouse 58 and double-clicking on the specific part of the X-ray two-dimensional radiographic image PI3. Further, if the operator single-clicks on any tomography image TI3, the position of that tomography image TI3 in the X-ray two-dimensional radiographic image PI3 is shown for example by blinking the relevant dotted line.

To sum up, the X-ray hybrid diagnosis system 100 according to the present embodiment is configured to obtain X-ray two-dimensional radiographic image(s) PI and tomography image(s) TI using CR unit 103 and CT unit 101, respectively, and the X-ray two-dimensional radiographic image(s) PI include a common landmark so that the operator can conduct a diagnosis easily and swiftly. For a patient who needs to have CR and CT scan images inspected, the CR and CT scan operations carried out for the patient lying on the same cradle 117 can reduce the strains placed on the patient due to movement. In cases where contrast medium need to be administered to the patient for CR and CT scan operations, the both operations can be performed with only one administration of the contrast medium, and thus the amount of contrast media can be reduced.

Image reconstruction for obtaining CT images according to the present embodiment may be implemented using a three-dimensional image reconstruction scheme by the feldkamp method known in the art. Alternatively, other three-dimensional image reconstruction method may be applied, and two-dimensional image reconstruction method may also be utilized. Image qualities for each part to be inspected may vary, for example, depending upon preferences of each operator. Therefore, the operator may be allowed to set the conditions for X-raying and imaging operations, which include optimum image quality of each part.

The method of CT scan operations consistent with the present embodiment is not limited to a specific scan mode. That is, the same advantages can be achieved with a conventional (axial) scan, cine scan, helical scan, variable pitch helical scan, or helical shuttle scan. The conventional scan is a scan mode in which X-ray tube 125 and X-ray detection unit 133 are rotated and projection data are acquired each time a cradle is moved in the Z-axis direction at regular pitches. The helical scan is a scan mode in which the projection data are acquired while X-ray tube 125 and X-ray detection unit 133 are rotated and the cradle 117 is moved at a constant speed. The variable pitch helical scan is a scan mode in which X-ray tube 125 and X-ray detection unit 133 are rotated like the helical scan mode but the projection data are acquired while the cradle 117 is moved at varied pitches. The helical shuttle scan is a scan mode in which the projection data are acquired like the helical scan mode while X-ray tube 125 and X-ray detection unit 133 are rotated but the cradle 117 reciprocates in the +Z direction and −Z direction. Further, it is to be understood that no limitation is placed with respect to the tilt angle of the gantry 101. Therefore, so-called ‘tilt scan’ mode is applicable and the same advantages can be achieved with tilted scanning gantry 101.

In the illustrated embodiments, medical X-ray hybrid diagnosis systems 100 with a CR unit and a CT unit combined together and incorporated therein have been described by way of example. However, the X-ray hybrid diagnosis system consistent with the present invention may be combined with any other systems; for example, X-ray CT-PET systems, and X-ray CT-SPECT systems may be embodied according to the present invention. Further, in the above-exemplified embodiments, the CR unit is described as a digital X-ray radiography system, but any analog X-ray radiography systems using a film may be adopted. In this instance, a scanner for converting the film into digital images may be provided.

It is contemplated that numerous modifications may be made to the exemplary embodiments of the invention without departing from the spirit and scope of the embodiments of the present invention as defined in the following claims.

What is claimed is:

1. An X-ray hybrid diagnosis system comprising:
an X-ray radiography unit irradiating a subject with X-rays from a first X-ray tube to obtain an X-ray two-dimensional radiographic image;
an X-ray CT unit irradiating the subject with X-rays from a second X-ray tube and acquiring projection data, to reconstruct an image using the acquired projection data, thereby obtaining a tomography image;
a control unit defining correspondences between position information of the subject located in the X-ray radiography unit and position information of the subject located in the X-ray CT unit.

2. The X-ray hybrid diagnosis system according to claim 1, further comprising a display unit displaying the X-ray two-dimensional radiographic image obtained in the X-ray radiography unit, wherein the position information of the subject located in the X-ray CT unit is shown in the displayed X-ray two-dimensional radiographic image.

3. The X-ray hybrid diagnosis system according to claim 1, further comprising a display unit displaying the tomography image obtained in the X-ray CT unit and the X-ray two-dimensional radiographic image obtained in the X-ray radiography unit concurrently, wherein the position information of the subject located in the X-ray CT unit is shown in the displayed X-ray two-dimensional radiographic image.

4. The X-ray hybrid diagnosis system according to claim 1, wherein the X-ray CT unit comprises a condition-setting unit in which conditions for obtaining the tomography image are set with the help of the X-ray two-dimensional radiographic image.

5. The X-ray hybrid diagnosis system according to claim 1, further comprising a mark member that is placed in a position relative to the subject, wherein the X-ray two-dimensional radiographic image is obtained in the X-ray radiography unit for the subject of which a target part is determined relative to the mark member, and the projection data are acquired in the X-ray CT unit for the subject of which a target range is determined relative to the mark member.

6. The X-ray hybrid diagnosis system according to claim 2, wherein the position information of the subject located in the X-ray CT unit shown in the X-ray two-dimensional radiographic image comprises a range of the subject for which the projection data are acquired in the X-ray CT unit.

7. The X-ray hybrid diagnosis system according to claim 2, wherein the X-ray two-dimensional radiographic image comprises a first X-ray two-dimensional radiographic image and a second X-ray two-dimensional radiographic image that is different from the first X-ray two-dimensional radiographic image; and
wherein a first tomography image corresponding to the first X-ray two-dimensional radiographic image and a second tomography image corresponding to the second two-dimensional radiographic image are displayed while the first X-ray two-dimensional radiographic image and the second X-ray two-dimensional radiographic image are displayed.

8. The X-ray hybrid diagnosis system according to claim 2, wherein the display unit includes a pointer for indicating a position in the X-ray two-dimensional radiographic image to display a specific tomography image corresponding to the indicated position of the X-ray two-dimensional radiographic image.

9. The X-ray hybrid diagnosis system according to claim 1, wherein the position information of the subject located in the X-ray radiography unit and the X-ray CT unit comprises information on a position along a body axis of the subject.

10. The X-ray hybrid diagnosis system according to claim 2, further comprising a display unit displaying the tomography image obtained in the X-ray CT unit and the X-ray two-dimensional radiographic image obtained in the X-ray radiography unit concurrently, wherein the position information of the subject located in the X-ray CT unit is shown in the displayed X-ray two-dimensional radiographic image.

11. The X-ray hybrid diagnosis system according to claim 2, wherein the X-ray CT unit comprises a condition-setting unit in which conditions for obtaining the tomography image are set with the help of the X-ray two-dimensional radiographic image.

12. The X-ray hybrid diagnosis system according to claim 2, further comprising a mark member that is placed in a position relative to the subject, wherein the X-ray two-dimensional radiographic image is obtained in the X-ray radiography unit for the subject of which a target part is determined relative to the mark member, and the projection data are acquired in the X-ray CT unit for the subject of which a target range is determined relative to the mark member.

13. The X-ray hybrid diagnosis system according to claim 6, wherein the X-ray two-dimensional radiographic image comprises a first X-ray two-dimensional radiographic image and a second X-ray two-dimensional radiographic image that is different from the first X-ray two-dimensional radiographic image; and
wherein a first tomography image corresponding to the first X-ray two-dimensional radiographic image and a second tomography image corresponding to the second two-dimensional radiographic image are displayed while the first X-ray two-dimensional radiographic image and the second X-ray two-dimensional radiographic image are displayed.

14. The X-ray hybrid diagnosis system according to claim 3, wherein the display unit includes a pointer for indicating a position in the X-ray two-dimensional radiographic image to display a specific tomography image corresponding to the indicated position of the X-ray two-dimensional radiographic image.

15. The X-ray hybrid diagnosis system according to claim 2, wherein the position information of the subject located in the X-ray radiography unit and the X-ray CT unit comprises information on a position along a body axis of the subject.

16. The X-ray hybrid diagnosis system according to claim 6, wherein the position information of the subject located in the X-ray radiography unit and the X-ray CT unit comprises information on a position along a body axis of the subject.

17. The X-ray hybrid diagnosis system according to claim 8, wherein the position information of the subject located in the X-ray radiography unit and the X-ray CT unit comprises information on a position along a body axis of the subject.
